(19) United States
(12) Patent Application Publication
(10) Pub. No.: US 2012/0141572 A1
(43) Pub. Date: Jun. 7, 2012

(54) Surface Coating for Biomedical Implants and Electrodes

(75) Inventors: Roland Hessler, Innsbruck (AT); Claude Jolly, Innsbruck (AT); Soeren Schilp, Innsbruck-Igls (AT); Volker Faust, Aachen (DE); Wolfgang Fischler, Innsbruck (AT)

(73) Assignee: MED-EL ELEKTROMEDIZINISCHE GERAETE GMBH, Innsbruck (AT)

(21) Appl. No.: 13/309,810

(22) Filed: Dec. 2, 2011

Related U.S. Application Data
(60) Provisional application No. 61/419,061, filed on Dec. 2, 2010.

(51) Int. Cl. A61B 17/00; A61N 5/06; C08F 2/00; A61K 47/30; B05D 3/00; A61F 11/04; A61N 1/05

(52) U.S. Cl. 424/422; 606/1; 607/137; 607/92; 427/2.24; 427/2.21

(57) ABSTRACT

An implantable device is described based on an implant having a textured outer surface of polymer material with limited transparency. A smooth outer polymer coating covers and cross-links with the textured outer surface to form a coated implant of greater transparency than the polymer material of the implant.

This application claims priority from U.S. Provisional Patent Application 61/419,061, filed Dec. 2, 2010, which is incorporated herein by reference.

### TECHNICAL FIELD

The present invention relates to implantable biomedical devices, and specifically, to surface coating on such devices and their electrodes.

### BACKGROUND ART

Implantable biomedical devices such as implant electrodes and electrical prosthesis can be implanted in the body to provide electrical stimulation to internal organs and tissue. For example, intra cochlear electrodes restore some sense of hearing by direct electrical stimulation of the neural tissue near an electrode contact. As more and more patients with significant and usable residual hearing are implanted with cochlear implants, it becomes important to use a minimally traumatic electrode. In addition, devices may be implanted in a subject when the subject is at a very young age and it may be necessary to re-implant several times during a lifetime. Each consecutive insertion of a cochlear implant may cause trauma to spiral ganglion cells to a minimum. Trauma to spiral ganglion cell is cumulative and cannot be undone in the present state of technology.

To reduce trauma to the organ or tissue and consequently to preserve a maximum of living tissue in the cochlea, implant electrodes should be soft and flexible, and insertion forces should be minimum. Unfortunately, most cochlear implant electrodes on the market today require significant force to be inserted, even for distances which are much less than the full length of the scala tympani. The required force to insert the implant electrode is related to various factors including the size, geometry, number of electrode contacts, internal structure and the material used in the fabrication of the particular device. Material used in such devices includes materials for wires, contacts, functional metallic or polymer segment, and bulk material. The size of the implant electrode, the rigidity of the material used, the hydrophobicity of the outer shell of the electrode material, the energy stored in one way or another in the electrode device and the insertion process of the device have an impact on the amount and location of damages that will be inflected to the tissue during electrode placement. In addition, removal and replacement of the system or of particular parts of the system may also cause trauma and damage to living tissue.

It has been proposed to coat implantable devices with a time released pharmacological substance having therapeutic properties to promote healing of the trauma. So far, this has been addressed using biodegradable polymers such as poly lactic acid, poly glycolic acid, and other biodegradable polymers mixed with some drug. But one problem with these biodegradable polymers is that even when very thin layers are applied they change the mechanical characteristic of the device, especially implant electrodes, by making the device extremely rigid. That is a significant issue. Another problem is that these polymers can degrade by releasing acidic compounds which change the pH of the surrounding tissue. Another problem is that the existing coatings are not bonded to the silicone material of electrode carrier, but simply surround the silicone without any adhesion and can easily rupture during device manipulations.

Another possible approach is to coat implantable devices using non-degrading biostable polymers as a drug eluting matrix instead of biodegradable polymers. These do not release degradation products or change the pH of the surrounding tissue, but they do remain permanently in the body and all known foreign tissue reactions like inflammation or other immunological reactions can occur.

It also has been proposed to reduce the insertion force and insertion trauma by coating the outside of the implant electrode with a lubricant coating that reduces the friction between the electrode device and the tissue it is entering. But a lubricant coating also makes the implant electrode more slippery and difficult for the surgeon to manipulate. And again, there are issues with the specific substances used as lubricants with regards to whether and how they breakdown after insertion.

After implantation of an implant device, the cells in the adjacent tissues are interrogating the biomaterial and generally encapsulate the foreign body with a fibrosis tissue shell. An electrode array that has been encapsulated in such a fibrous tissue shell suffers from increased electrical impedance. That increases the power consumption of the implant system. This can be particularly problematic for completely implantable systems which require very low power consumption. The hearing performance of the patient might be not as good as it could or should be due to the increased electrical impedance of the electrode array. Smooth implant surfaces can minimize this adverse encapsulation process.

In addition to tissue trauma, insertion of the implant electrode creates significant forces within the device itself. Within the resilient silicone material of the electrode carrier are delicate metal electrode contacts that apply the electrical stimulation signals to adjacent tissues and connecting wires that deliver the stimulation signals to the electrode contacts. These structures are quite small and fragile. They can become damaged and broken either during manufacturing, shipping, handling or even post-surgically. Any damage to the wires and/or contacts typically cannot be repaired and degrades the performance of the implant device. Thus one important aspect of the manufacturing, shipping and surgical processes is performing quality control checks to inspect and verify the integrity of the implant electrode and its internal structures. For example, wire breakages, possible short circuits or other failures can be visually detected under a microscope.

But even though the silicone material of the electrode device is naturally transparent, the production molding process leaves the outer surface with textured features that obscure visual inspection of the interior of the device. The transparency of the device can be temporarily improved for manufacturing quality assurance inspection by dipping the device in water, but this is only a temporary effect. Thus when the surgeon removes the implant device from its shipping packaging for surgical implantation, the interior of the device is once again obscured.

### SUMMARY

Embodiments of the present invention are directed to an implantable device such as a cochlear implant electrode and/or an implantable optical stimulation device. An implant body of polymer material with limited transparency has a textured outer surface. A smooth outer polymer coating covers and cross-links with the textured outer surface to form a coated implant of greater transparency than the polymer material.

The polymer material and the polymer coating may be substantially the same material, for example, a silicone material. This way the polymer material and the polymer coating can have substantially similar strength and flexibility. In some embodiments, the polymer coating may include a time releasable pharmaceutical substance. At least a portion of the polymer coating may have an optical tint of a different color than the polymer material of the implant. In some embodiments, the polymer coating may include a time releasable pharmaceutical substance.

Embodiments of the present invention also include a method of forming an implantable device such as a cochlear implant electrode and/or an implantable optical stimulation device. An implant body is produced of polymer material with limited transparency having a textured outer surface. Then a smooth outer polymer coating is applied that covers and cross-links with the textured outer surface to form a coated implant of greater transparency than the polymer material.

In further such embodiments, the polymer material and the polymer coating may be substantially the same material, for example, a silicone material. The polymer material and the polymer coating may have substantially similar strength and flexibility. In some embodiments, the polymer coating may include a time releasable pharmaceutical substance. At least a portion of the polymer coating may have an optical tint of a different color than the polymer material of the implant.

The smooth outer polymer coating may be applied by a spray coating technique, a dip coating technique or other techniques. The electrode contacts can be masked for the coating process.

### BRIEF DESCRIPTION OF THE DRAWINGS

FIG. 1 shows an implant electrode having an outer polymer coating according to an embodiment of the present invention.

FIG. 2 A-E shows cross-sectional views of implant electrodes having various specific coating structures according to embodiments of the present invention.

FIG. 3 shows a view of a section of an uncoated cochlear implant electrode.

FIG. 4 shows a view of a section of a cochlear implant electrode having an outer coating according to an embodiment of the present invention.

### DETAILED DESCRIPTION

Embodiments of the present invention are directed to an implantable device such as a cochlear implant electrode and/or an implantable optical stimulation device which has a textured outer surface of polymer material with limited transparency. For example, typically a cochlear implant electrode includes a silicone carrier material which encapsulates multiple stimulation contacts and connecting wires. A smooth outer polymer coating covers and cross-links with the textured outer surface of the device to form a coated implant having greater transparency than the polymer material of the implant.

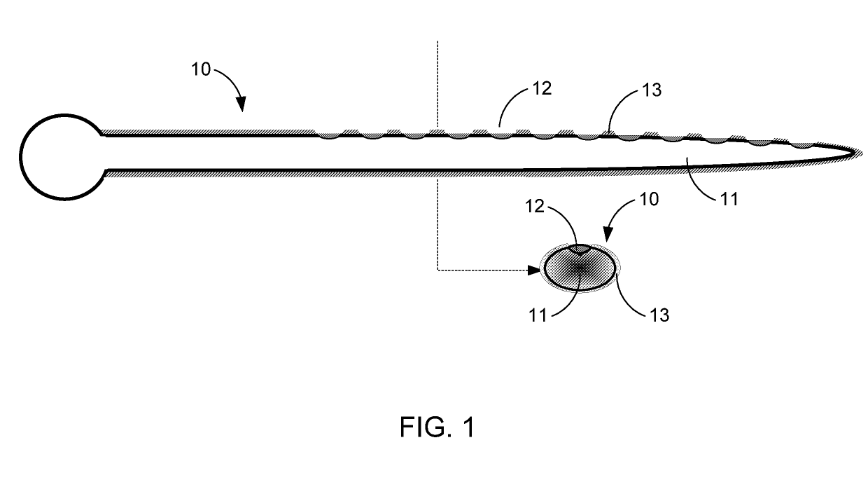

FIG. 1 shows an example of a cochlear implant electrode 10 according to one specific embodiment of the present invention. An elastomeric electrode carrier 11 (e.g. of molded silicone) of polymer material with limited transparency has a textured outer surface. The electrode carrier 11 also encapsulates multiple electrode contacts 12, typically in its distal section which is called the electrode array, for electrically stimulating cochlear tissue and connecting wires which deliver the stimulation signals to the electrode contacts 12. A smooth outer coating 13 covers and cross-links with the textured outer surface of at least a portion of the electrode carrier 11 to form a coated cochlear implant electrode 10 having greater transparency than the untreated polymer material of the electrode carrier 11. The outer coating 13 is substantially the same material as the electrode carrier 11, e.g., a silicone material such that the electrode carrier 11 and the outer coating 13 can have substantially similar strength and flexibility. At least a portion of the outer coating 13 may have an optical tint of a different color than the material of the electrode carrier 11 to highlight specific features or portions of the implant electrode 10. In a specific embodiment only the electrode array section may be coated.

In some embodiments, the outer coating 13 may include a therapeutically effective amount of a pharmaceutical substance which is releasable over time into the surrounding tissue of the inner ear. The concentration of the pharmaceutical substance in the outer coating 13 may need to be relatively high to be effective in the cochlear fluids, especially in the more apical parts of the cochlea where still functioning hair cells may reside. Examples of pharmaceutical substances which may be usefully incorporated into the outer coating 13 include antibiotics and/or steroids (e.g., dexamethasone)_{[w1]}, which elute from the outer coating 13 over time until depletion of the substance. The release rate of the pharmaceutical substance from the outer coating 13 is a function of the drug ratio in the coating, the thickness of the coating, and the specific process by which it is applied.

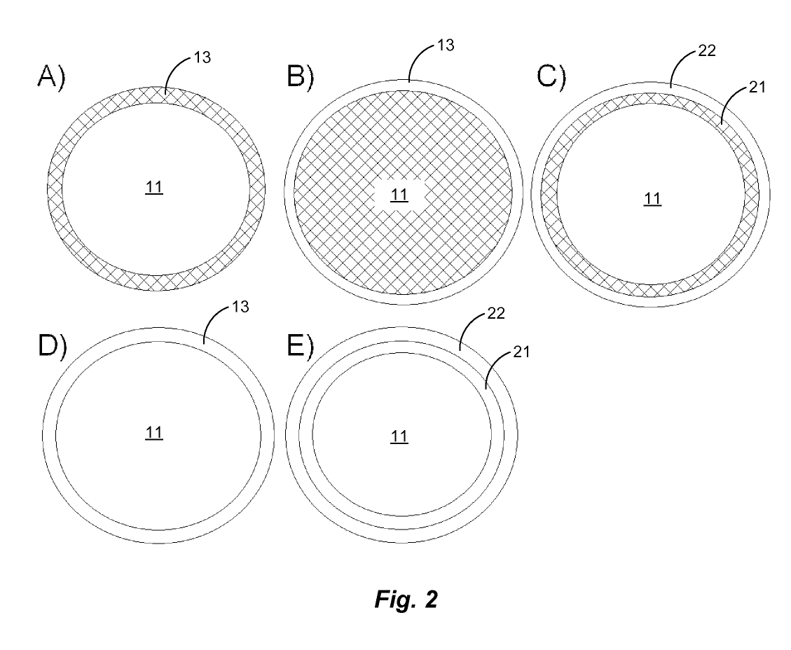

FIG. 2 A-E shows cross-sectional views of implant electrodes having various specific coating structures according to embodiments of the present invention. While the outer coating 13 is made from the same material as the electrode carrier 11 as shown in FIG. 2D, in some embodiments the structures of the two can be different, for example dense or porous. The porosity of the outer coating 13 has a strong influence on the drug release characteristics of the device. FIG. 2A shows an embodiment having a relatively dense electrode carrier 11 with a relatively porous the outer coating 13 incorporating a pharmaceutical substance. Elution of the pharmaceutical substance from the outer coating 13 takes place after hydration of the cochlear implant electrode 10 in extra cellular fluids. One advantage of a porous outer coating 13 is to increase the surface area and thereby the release rate of the pharmaceutical substance. In other embodiments, it may be useful to have a dense outer coating 13 with a porous electrode carrier 11 as shown in FIG. 2B.

Some embodiments may be based on a multilayer arrangement such as shown in FIG. 2C where a dense inner electrode carrier 11 is covered with two outer layers: a first porous outer coating 21 covered by dense second outer coating 22, where one of the outer coatings may hold a pharmaceutical substance, while the other outer coating has either a second pharmaceutical substance, or none. Or the concentrations of a single pharmaceutical substance may be different in the different outer coatings.

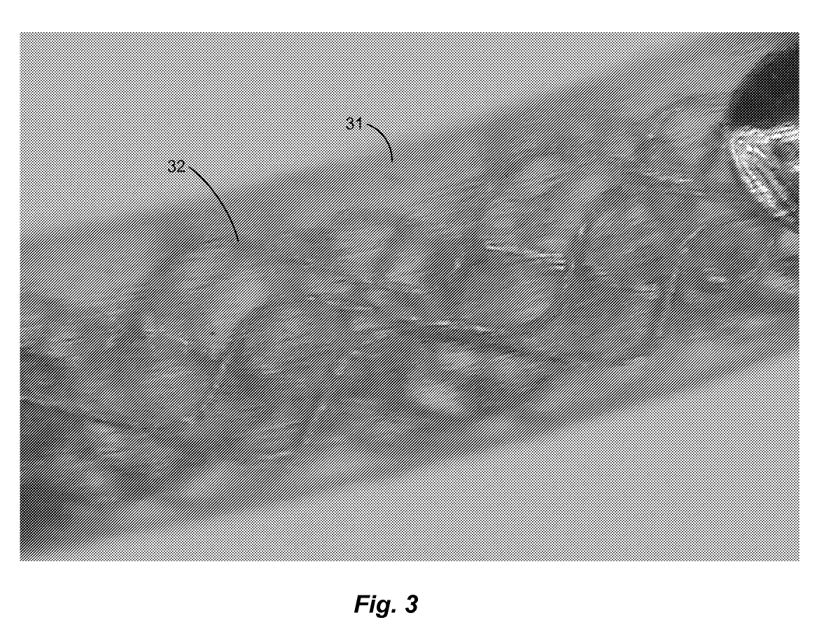

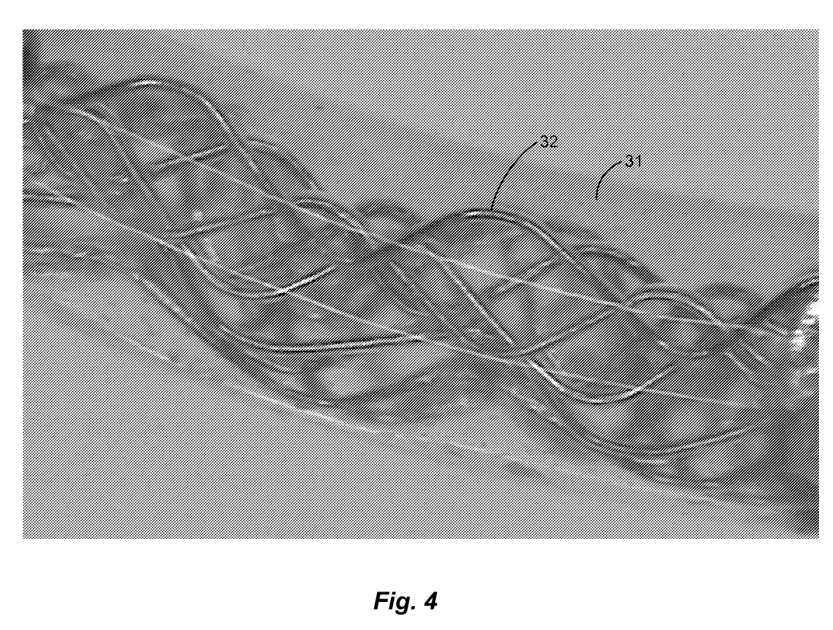

In FIG. 3 one can see into the interior of an uncoated silicone electrode carrier 31, but the textured outer surface (an imprint effect from the production molding process) provides only limited transparency leaving unclear the structural details of the internal electrode wires 32. When the outer surface of the electrode carrier 31 is coated with a thin silicone outer layer, the texture features are filled in to form a smooth surface with much improved transparency as shown in FIG. 4 that allows for much easier visual inspection of the interior electrode wires 32. Moreover, the improved transparency effect is a lasting one which endures through shipping and handling, allowing the surgeon to easily verify the integrity of the implant electrode 10 immediately prior to implantation.

The outer coating 13 may be applied to the electrode carrier 11 in various ways. For example, the electrode carrier 11 may be immersed into a container of the coating material to cover it by a dip coating technique. Or the coating material may be sprayed onto the surface of the electrode carrier 11 to form the outer coating 13. The outer coating 13 can be as thin as just a few microns up to hundreds of microns thick using multiple application passes. An ultra thin outer coating 13 can be easily deposited with spray coating of the electrode carrier 11. Spray coating the outer layer 13 can use a silicone which has been diluted with a solvent, and the solvent can receive the pharmaceutical substance which can be dissolved in such a medium. Spray coating parameters can form porous or non-porous silicone outer layer 13. The size of the coated particles may be nanometer sized or μm-sized droplets.

In addition or alternatively, a masking technique may be used to confine the outer coating 13 to the desired portion of the surface of the electrode carrier 11. For example, it may be desirable to mask over the electrode contacts 12 while the outer coating 13 is applied, and then remove the protective masking afterwards. Masking can be accomplished by using clips, clips and foam pads, glucose drops, drops of polymer solution, or a shield between spray coater and parts of the implant electrode 10. Demasking can take place while the spray coated outer layer 13 is still in a wet state thereby limiting the rough edges at the border of coated/non coated part of the implant electrode 10.

As briefly mentioned above, it is advantageous for the critical physical parameters of the implant electrode 10 not be changed by the outer coating 13. Using the same silicone material for the polymer component of the outer coating 13 as already used for the electrode carrier 11 is advantageous related to mechanical characteristics, biocompatibility, biological efficiency, and adhesion between outer coating 13 and the electrode carrier 11 which leaves the implant electrode 10 unchanged in appearance, mechanical and dimensional characteristics or surface properties like wettability or surface charge. Using the same material of the electrode carrier 11 and the outer coating 13 material results in matching surface characteristics such as hydrophobicity and electrical charging, and thereby maximizing the intermolecular forces between both components to result in good adhesion between them.

Having a small amount of pharmaceutical substance in the outer coating 13 (typically 0.1-10%) has little if any effect on the mechanical properties of the implant electrode 10. And using the same polymer matrix in the outer coating 13 as the polymer of the electrode carrier 11 means that the coating matrix has no additional influence on the biocompatibility of the device as a whole. No additional impurities or degradation products occur and only the pharmaceutical substance has any meaningful biological activity on the surrounding tissue.

Moreover, because the surface of the outer coating 13 is smoother than the textured surface of the electrode carrier 11, there will be reduced tissue trauma during surgical insertion of the device. Similarly, there should also be less tissue growth after insertion, and less adhesion of proteins (e.g. particles of the blood) during and after insertion

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable device comprising:
an implant body of polymer material with limited transparency having a textured outer surface; and
a smooth outer polymer coating covering and cross-linking with the textured outer surface to form a coated implant body of greater transparency than the polymer material.

2. An implantable device according to claim 1, wherein the polymer material and the polymer coating are substantially the same material.

3. An implantable device according to claim 2, wherein the material is a silicone material.

4. An implantable device according to claim 1, wherein the polymer material and the polymer coating have substantially similar strength and flexibility.

5. An implantable device according to claim 1, wherein the polymer coating includes a time releasable pharmaceutical substance.

6. An implantable device according to claim 1, wherein at least a portion of the polymer coating has an optical tint of a different color than the polymer material of the implant.

7. An implantable device according to claim 1, wherein the device is a cochlear implant electrode.

8. An implantable device according to claim 1, wherein the device is an implantable optical stimulation device.

9. A method of forming an implantable device comprising:
producing an implant body of polymer material with limited transparency having a textured outer surface; and
applying a smooth outer polymer coating covering and cross-linking with the textured outer surface to form a coated implant body of greater transparency than the polymer material.

10. A method according to claim 9, wherein the polymer material and the polymer coating are substantially the same material.

11. A method according to claim 10, wherein the material is a silicone material.

12. A method according to claim 9, wherein the polymer material and the polymer coating have substantially similar strength and flexibility.

13. A method according to claim 9, wherein the polymer coating includes a time releasable pharmaceutical substance.

14. A method according to claim 9, wherein at least a portion of the polymer coating has an optical tint of a different color than the polymer material of the implant.

15. A method according to claim 9, wherein the device is a cochlear implant electrode.

16. A method according to claim 9, wherein the device is an implantable optical stimulation device.

17. A method according to claim 9, wherein a contact masking technique is used for applying the smooth outer polymer coating.

18. A method according to claim 9, wherein a spray coating technique is used for applying the smooth outer polymer coating.

19. A method according to claim 9, wherein a dip coating technique is used for applying the smooth outer polymer coating.
